(19) United States
(12) Patent Application Publication
(10) Pub. No.: US 2020/0316110 A1
(43) Pub. Date: Oct. 8, 2020

(54) METHOD FOR BONE TISSUE REGENERATION IN EXPERIMENTS

(71) Applicant: OOO "FELIX", Respublika Tatarstan (RU)

(72) Inventor: Fedor Vladimirovich DEVYATOV, Kazan (RU)

(21) Appl. No.: 16/904,986

(22) Filed: Jun. 18, 2020

Related U.S. Application Data
(63) Continuation of application No. 14/400,273, filed on Nov. 10, 2014, now abandoned.

(30) Foreign Application Priority Data
May 10, 2012 (RU) 2012119192

(51) Int. Cl. A61K 33/24; A61K 31/663; A61K 33/14; A61L 24/02; A61L 24/00; A61K 9/00; A61K 47/02; A61K 9/08

(52) U.S. Cl. CPC A61K 33/24; A61K 31/663; A61K 33/14; A61L 24/02; A61L 2430/02; A61K 9/0019; A61K 47/02; A61K 9/08; A61L 24/0015

(57) ABSTRACT

The invention relates to the treatment of different bone injuries, particularly fractures and fissure fractures. In order to reduce the time taken for bone tissue to regenerate at the site of damage, a method is used for regenerating bone tissue by fixing fragments of the damaged bone with a plaster cast or a bandage made of a polymer material and introducing into the fracture zone an aqueous solution containing 1-hydroxyethylidene diphosphonic acid in an amount of (1.80-2.06) g/l, anhydrous calcium chloride in an amount of (1.44-2.22) g/l, gadolinium (III) nitrate hexahydrate in an amount of (0.30-0.40) g/l and dysprosium (III) chloride hexahydrate in an amount of (0.038-0.076) g/l, with a pH of (7.3-7.8), wherein, prior to being introduced into the fracture zone, the above solution is brought to a temperature of (30-100)° C., is held at this temperature for (1-48) hours and is then cooled to room temperature.

The present invention relates to the treatment of different bone injures, specifically, to fractures and fissure fractures, and can be used in medical and veterinary therapy and surgery.

A bone tissue regeneration method, wherein the injured bone fragments are immobilized with a plaster cast and subsequent introduction of an aqueous solution comprising 1-hydroxyethylidene diphosphonic acid, calcium chloride, and gadolinium (III) nitrate into the bone fracture zone at room temperature, is known in the art [1]. The disadvantage of said method is relatively long bone tissue regeneration time.

A method comprising the cumulative features and the attained technical effect, which are most closely related to the object of the present invention, is the method for bone tissue regeneration in experiments comprising immobilization of the injured bone fragments with a plaster cast or plastic bandage and subsequent introduction of an aqueous solution with pH 7.3-7.8, comprising 1.80-2.06 g/L of 1-hydroxyethylidene diphosphonic acid, 1.44-2.22 g/L of anhydrous calcium chloride, 0.30-0.40 g/L of gadolinium (III) nitrate hexahydrate, and 0.038-0.076 g/L of dysprosium (III) chloride hexahydrate, into the bone fracture zone at room temperature [2]. The disadvantage of said method, taken as a prototype, is also relatively long bone tissue regeneration time.

The objective of the present invention is to further reduce the damaged or defected bone tissue regeneration time as well as time required to restore normal physiological function of the injured bone.

The stated objective is achieved as follows: in the existing method for bone tissue regeneration in experiments, wherein the injured bone fragments are immobilized with a plaster cast or plastic bandage, the aqueous solution with pH 7.3-7.8 comprising 1.80-2.06 g/L of 1-hydroxyethylidene diphosphonic acid, 1.44-2.22 g/L of anhydrous calcium chloride, 0.30-0.40 g/L of gadolinium (III) nitrate hexahydrate, and 0.038-0.076 g/L of dysprosium (III) chloride hexahydrate is injected into the bone fracture zone; however, prior to the injection thereof into the fracture zone, said solution is brought to 30° C.-100° C., kept at said temperature for 1-48 hrs., and then brought back to room temperature. Employing said method of the present invention results in significant acceleration of bone tissue regeneration and further reduction (by 15-20%) of time required to restore normal physiological function of the injured bone in comparison to the method described in prototype [2].

No method for bone tissue regeneration in experiments, wherein all features thereof would fully match the features of the present invention, has ever been disclosed in the literature. Thus, the present invention can be claimed to comply with the first criterion of the Russian Federation Patent Law, novelty. The significant time reduction in restoration of normal physiological function of the injured bone, which was observed in the experiment, relative to existing method [2], cannot be deduced from the comparison of the features of prototype [2] with the altered features, i.e., a prior heating of the solution used therein specifically to the aforementioned 30° C.-100° C. temperature and subsequent keeping said solution at said temperature specifically over the aforementioned (1-48 hrs.). Therefore, the claimed technical solution does not clearly follow from the prior art and thus, it complies with the second criterion of the Russian Federation Patent Law, inventive level. The claimed method of the present invention does not require any special equipment and can be easily realized even in an outpatient setting, thus, it also complies with the third criterion of the Russian Federation Patent Law, industrial applicability.

The claimed method for bone tissue regeneration in experiments can be further illustrated with the following examples:

### EXAMPLE 1 (PREPARATION OF THE CLAIMED SOLUTION)

A 1000 ml volumetric flask is filled with 1.80-2.06 g/L of 1-hydroxyethylidene diphosphonic acid, 1.44-2.22 g/L of anhydrous calcium chloride, 0.30-0.40 g/L of gadolinium (III) nitrate hexahydrate, 0.038-0.076 g/L of dysprosium (III) chloride hexahydrate, and 950 ml of distilled water. pH of the resulting mixture is brought to 7.3-7.8 with any concentrated alkaline solution (such as 10% aqueous sodium hydroxide solution). The resulting solution is brought to 1000 ml with distilled water, heated to 30° C.-100° C., kept at that temperature for 1-48 hrs., and then cooled down to room temperature (20-25° C.). The resulting solution is then used in the claimed method.

### EXAMPLE 2

An aqueous solution with the following composition (g/L):

| | |
|---|---|
| 1-hydroxyethylidene diphosphonic acid | 1.80 |
| Anhydrous calcium chloride | 1.44 |
| Gadolinium (III) nitrate hexahydrate | 0.30 |
| Dysprosium (III) chloride hexahydrate | 0.038 |

is prepared with pH 7.3, brought to 30° C., kept at said temperature for 48 hrs., then cooled to room temperature. Under anesthesia, 2 ml of the prepared solution are then injected over a period of 2 min. into the injured bones of a Northern European cat with lower hind leg fractures, which were induced one hour prior to the experiment. After injection, the fractured bone sites are immobilized in a plaster cast or bandaged with plastic bandages, and the injured leg segments are kept as such until recovery of locomotor function. Regeneration of the injured bone tissue is followed with X-rays, using a radiotransparent synthetic Lohmann&Rauscher Cellacast Xtra bandage or cutting out special “windows” around the injury site. Bone tissue regeneration time is determined visually, based on the injured animal's behavior (from the time of injection of the solution until the time of full locomotor function recovery of the injured bone with ±12 hrs. accuracy). The indicator for this particular case study is shown in Table 1.

### EXAMPLE 3

Follows the general procedure of Example 2, but for injection, using 2 ml of the aqueous solution with the following composition, g/L:

| | |
|---|---|
| 1-hydroxyethylidene diphosphonic acid | 1.92 |
| Anhydrous calcium chloride | 1.88 |
| Gadolinium (III) nitrate hexahydrate | 0.35 |
| Dysprosium (III) chloride hexahydrate | 0.055 |

with pH 7.5, which is then heated to 60° C., and kept at this temperature for 6 hrs. Bone tissue regeneration time for this case study is shown in Table 1.

### EXAMPLE 4

Same as Example 2, but for injection, using 2 ml of the aqueous solution with the following composition, g/L:

| | |
|---|---|
| 1-hydroxyethylidene diphosphonic acid | 1.92 |
| Anhydrous calcium chloride | 1.88 |
| Gadolinium (III) nitrate hexahydrate | 0.35 |
| Dysprosium (III) chloride hexahydrate | 0.055 |

with pH 7.7, which is then heated to 70° C., and kept at this temperature for 4 hrs. Bone tissue regeneration time for this case study is shown in Table 1.

### EXAMPLE 5

Follows the general procedure of Example 2, but for injection, using 2 ml of the aqueous solution with the following composition, g/L:

| | |
|---|---|
| 1-hydroxyethylidene diphosphonic acid | 2.06 |
| Anhydrous calcium chloride | 2.20 |
| Gadolinium (III) nitrate hexahydrate | 0.40 |
| Dysprosium (III) chloride hexahydrate | 0.076 |

with pH 7.8, which is then heated to 100° C. and kept at this temperature for 1 hr.

### EXAMPLE 6

Same as Example 2, but for injection, using 2 ml of the aqueous solution with the following composition, g/L:

| | |
|---|---|
| 1-hydroxyethylidene diphosphonic acid | 1.80 |
| Anhydrous calcium chloride | 1.44 |
| Gadolinium (III) nitrate hexahydrate | 0.30 |
| Dysprosium (III) chloride hexahydrate | 0.038 |

with pH 7.5, which is then heated to 70° C., and kept at this temperature for 4 hrs. Bone tissue regeneration time for this case study is shown in Table 1.

### EXAMPLE 7

Same as Example 2, but for injection, using 2 ml of the aqueous solution with the following composition, g/L:

| | |
|---|---|
| 1-hydroxyethylidene diphosphonic acid | 1.80 |
| Anhydrous calcium chloride | 1.44 |
| Gadolinium (III) nitrate hexahydrate | 0.30 |
| Dysprosium (III) chloride hexahydrate | 0.038 |

with pH 7.8, which is then heated to 70° C., and kept at this temperature for 4 hrs. Bone tissue regeneration time for this case study is shown in Table 1.

### EXAMPLE 8 (COMPARATIVE)

Same as Example 3, but with pH of the solution at 7.0, Bone tissue regeneration time for this case study is shown in Table 1.

### EXAMPLE 9 (COMPARATIVE)

Same as Example 3, but with pH of the solution at 8.0. Bone tissue regeneration time for this case study is shown in Table 1.

### EXAMPLE 10 (COMPARATIVE)

Same as Example 5, but the solution is kept at the temperature mentioned therein for 0.5 hrs. Bone tissue regeneration time for this case study is shown in Table 1.

### EXAMPLE 11 (COMPARATIVE)

Same as Example 2, but the solution is kept at the temperature mentioned therein for 60 hrs. Bone tissue regeneration time for this case study is shown in Table 1.

### EXAMPLE 12 (ANALOGOUS TO [1])

Follows the general procedure of Example 2, but for injection, using 2 ml of the aqueous solution with the following composition, g/L:

| | |
|---|---|
| 1-hydroxyethylidene diphosphonic acid | 2.00 |
| Anhydrous calcium chloride | 2.20 |
| Gadolinium (III) nitrate hexahydrate | 0.40 |

with pH 8.0, and omitting the steps of heating the solution to 30° C. and keeping it at said temperature. Bone tissue regeneration time for this case study is shown in Table 1.

### EXAMPLE 13 (USING [2] AS A PROTOTYPE)

Same as Example 4, using the same composition of the solution and same pH, but omitting the steps of bringing the solution to 70° C. and keeping it at said temperature. Bone tissue regeneration time for this case study is shown in Table 1.

### EXAMPLE 14 (USING [2] AS A PROTOTYPE)

Same as Example 4, using the same composition of the solution and same pH, but omitting the steps of heating the solution to 70° C. and keeping it at said temperature. Bone tissue regeneration time for this case study is shown in Table 1.

### EXAMPLE 15 (USING [2] AS A PROTOTYPE)

Same as Example 5, using the same composition of the solution and same pH, but omitting the steps of heating the solution to 100° C. and keeping it at said temperature. Bone tissue regeneration time for this case study is shown in Table 1.

TABLE 1

| | | Time to restore full locomotor |
|---|---|---|
| Example | Injured | function of the injured bone |
| # | Animal | segment, days |
| 2 | Northern | 5.0 |
| | European | |
| | Cat | |
| 3 | Northern | 4.5 |
| | European | |
| | Cat | |
| 4 | Northern | 4.5 |
| | European | |
| | Cat | |
| 5 | Northern | 5.0 |
| | European | |
| | Cat | |
| 6 | Northern | 4.5 |
| | European | |
| | Cat | |
| 7 | Northern | 5.0 |
| | European | |
| | Cat | |
| 8 | Northern | 6.0 |
| (com- | European | |
| parative) | Cat | |
| 9 | Northern | 6.5 |
| (com- | European | |
| parative) | Cat | |
| 10 | Northern | 6.0 |
| (com- | European | |
| parative) | Cat | |
| 11 | Northern | 4.5 |
| (com- | European | |
| parative) | Cat | |
| 12 | Northern | 9.0 |
| (analog) | European | |
| | Cat | |
| 13 | Northern | 6.0 |
| (proto- | European | |
| type) | Cat | |
| 14 | Northern | 6.5 |
| (proto- | European | |
| type) | Cat | |
| 15 | Northern | 6.0 |
| (proto- | European | |
| type | Cat | |

### EXAMPLE 16

An aqueous solution with the following composition (g/L):

| | |
|---|---|
| 1-hydroxyethylidene diphosphonic acid | 1.80 |
| Anhydrous calcium chloride | 1.44 |
| Gadolinium (III) nitrate hexahydrate | 0.30 |
| Dysprosium (III) chloride hexahydrate | 0.038 |

is prepared with pH 7.3, then brought to 30° C., and kept at said temperature for 48 hrs., after which it is cooled to room temperature. Under anesthesia, the prepared solution is then administered to a rabbit of unspecified breed, whose both femurs have been injured by drilling with an electric drill, 5 mm in diameter, under general anesthesia. However, in this experiment, 1 ml of said solution (over a period of 2 min., as in Example 2) is injected into the injury site of only one of the injured femurs (the second analogous bone is used as control). After that, all injury sites are immobilized in a plaster cast or bandaged with plastic bandages, and followed with X-rays for bone tissue regeneration, same as in Example 2. Bone tissue regeneration time is determined visually, based on the injured animal's behavior (from the time of injection of the solution until the time of full recovery of locomotor function of the injured bone with ±12 hrs. accuracy). The indicator for this particular case study is shown in Table 2.

### EXAMPLE 17

Follows the general procedure of Example 16, but for injection, using the aqueous solution prepared in the same composition and by the same procedure as described in Example 3.

### EXAMPLE 18

Follows the general procedure of Example 16, but using the aqueous solution for injection that has been prepared in the same composition and by the same procedure as described in Example 4.

### EXAMPLE 19

Follows the general procedure of Example 16, but for injection, using the aqueous solution prepared in the same composition and by the same procedure as described in Example 5.

### EXAMPLE 20

Follows the general procedure of Example 16, but for injection, using the aqueous solution prepared in the same composition and by the same procedure as described in Example 6.

### EXAMPLE 21

Follows the general procedure of Example 16, but for injection, using the aqueous solution prepared in the same composition and by the same procedure as described in Example 7.

### EXAMPLE 22 (COMPARATIVE)

Follows the general procedure of Example 16, but for injection, using the aqueous solution prepared in the same composition and by the same procedure as described in Example 8.

### EXAMPLE 23 (COMPARATIVE)

Follows the general procedure of Example 16, but for injection, using the aqueous solution prepared in the same composition and by the same procedure as described in Example 9.

### EXAMPLE 24 (COMPARATIVE)

Follows the general procedure of Example 16, but for injection, using the aqueous solution prepared in the same composition and by the same procedure as described in Example 10.

### EXAMPLE 25 (COMPARATIVE)

Follows the general procedure of Example 16, but for injection, using the aqueous solution prepared in the same composition and by the same procedure as described in Example 11.

### EXAMPLE 26 (ANALOGOUS TO [1])

Follows the general procedure of Example 16, but for injection, using the aqueous solution prepared in the same composition and by the same procedure as described in Example 12.

### EXAMPLE 27 (PROTOTYPE [2])

Follows the general procedure of Example 16, but for injection, using the aqueous solution prepared in the same composition and by the same procedure as described in Example 13.

### EXAMPLE 28 (PROTOTYPE [2])

Follows the general procedure of Example 16, but for injection, using the aqueous solution prepared in the same composition and by the same procedure as described in Example 14.

### EXAMPLE 29 (PROTOTYPE [2])

Follows the general procedure of Example 16, but for injection, using the aqueous solution prepared in the same composition and by the same procedure as described in Example 15.

Data comparing regeneration times of bone tissue, which have been previously injected with solutions as described in Examples 16-29, and regeneration times of bone tissue that haven't been injected with said solutions are shown in Table 2.

TABLE 2

| | | Time to restore full locomotor | |
|---|---|---|---|
| | | function of the injured bone | |
| | | segment, days | |
| | | For the bone | |
| Example | Injured | injected with | |
| # | Animal | the solution | For control |
| 16 | Unspecified | 4.0 | 18.0 |
| | Breed | | |
| | Rabbit | | |
| 17 | Unspecified | 3.5 | 17.5 |
| | Breed | | |
| | Rabbit | | |
| 18 | Unspecified | 4.0 | 18.0 |
| | Breed | | |
| | Rabbit | | |
| 19 | Unspecified | 4.0 | 18.0 |
| | Breed | | |
| | Rabbit | | |
| 20 | Unspecified | 4.0 | 17.5 |
| | Breed | | |
| | Rabbit | | |
| 21 | Unspecified | 3.5 | 18.0 |
| | Breed | | |
| | Rabbit | | |
| 22 | Unspecified | 4.5 | 18.0 |
| (com- | Breed | | |
| parative) | Rabbit | | |
| 23 | Unspecified | 4.5 | 17.5 |
| (com- | Breed | | |
| parative) | Rabbit | | |
| 24 | Unspecified | 4.5 | 17.5 |
| (com- | Breed | | |
| parative) | Rabbit | | |
| 25 | Unspecified | 4.0 | 18.0 |
| (com- | Breed | | |
| parative) | Rabbit | | |
| 26 | Unspecified | 8.5 | 18.0 |
| (analog) | Breed | | |
| | Rabbit | | |
| 27 | Unspecified | 5.0 | 17.5 |
| (proto- | Breed | | |
| type) | Rabbit | | |
| 28 | Unspecified | 5.0 | 18.0 |
| (proto- | Breed | | |
| type) | Rabbit | | |
| 29 | Unspecified | 5.5 | 18.5 |
| (proto- | Breed | | |
| type) | Rabbit | | |

### EXAMPLE 29

An aqueous solution with the same composition as that of Example 3 is prepared following the same procedure. Under anesthesia, 2.5 ml of said solution are administered over a 2 min. period to a stray mutt 6 hrs. after inducing a significantly displaced right front humeral fracture. After that, the injury site is immobilized in a plaster cast or bandaged with plastic bandages. Bone tissue regeneration is followed as described in Example 2. Bone tissue regeneration time is determined visually, based on the injured animal's behavior (from the time of injection of the solution until the time of full recovery of locomotor function of the injured bone with ±12 hrs. accuracy). The indicator for this particular case study is shown in Table 3.

### EXAMPLE 31

Follows the general procedure of Example 30, but for injection, using the aqueous solution prepared in the same composition and by the same procedure as described in Example 4.

### EXAMPLE 32 (COMPARATIVE)

Follows the general procedure of Example 30, but for injection, using the aqueous solution prepared in the same composition and by the same procedure as described in Example 10.

### EXAMPLE 33 (COMPARATIVE)

Follows the general procedure of Example 30, but for injection, using the aqueous solution prepared in the same composition and by the same procedure as described in Example 11.

### EXAMPLE 28 (PROTOTYPE [2])

Follows the general procedure of Example 30, but for injection, using the aqueous solution prepared in the same composition and by the same procedure as described in Example 14.

### EXAMPLE 35 (PROTOTYPE [2])

Follows the general procedure of Example 30, but for injection, using the aqueous solution prepared in the same composition and by the same procedure as described in Example 15.

Bone tissue regeneration time data when using technologies described in Examples 30-35 are shown in Table 3.

TABLE 3

| Ex- | | Time to restore full locomotor |
|---|---|---|
| ample | Injured | function of the injured bone |
| # | Animal | segment, days |
| 30 | Stray mutt | 7.5 |
| 31 | Stray mutt | 8.0 |
| 32 | Stray mutt | 9.0 |
| (com- | | |
| parative) | | |
| 33 | Stray mutt | 8.0 |
| (com- | | |
| parative) | | |
| 34 | Stray mutt | 10.5 |
| (proto- | | |
| type) | | |

The data in Tables 1-3 clearly demonstrate that the method of the present invention significantly reduces the time of bone tissue regeneration at the injury site (20-25%) as compared to prototype method [2], and said reduction was observed in all injured animals irrespective of the type. Equally important are the temperature range (30-100° C.) and the thermostating time (1-48 hrs.) at any of the temperatures of said range, as well as the solution's pH (7.3-7.8); and going outside of either lower or upper limit of said range usually results in increased bone tissue regeneration time (keeping the solution at the specified temperature longer shows no real changes in the earlier achieved indicators; therefore, increasing the time beyond 48 hrs. does not really lead to any significant changes). Our measurements of the regenerated bone tissue's strength showed no noticeable differences observed when using methods known in the art [1,2] or the regeneration method of the present invention. The blood count data of the animals during regeneration of the injured bone tissue and observation of the injured animals' subjective states during the treatment thereof (appetite, response, etc.) did not show any expressive toxicity of the claimed solution for injection; and also, none of the ingredients comprising said solution for injection is toxic, as per [3,4]. In conclusion, the claimed solution has a long shelf life and can be kept for a long time (at least 1 year) in a sealed container with no loss in the performance thereof.

### Literature

What is claimed is:

1. A method for bone tissue regeneration in experiments, comprising:
heating a solution for 25-48 hours at a temperature 30°-100° C., then cooling it for at least 2 hours to a temperature 15°−25° C.;
immobilizing of bone fragments with a plaster cast and
subsequent introducing of the solution into a bone fracture zone,
thus achieving the time reduction of the bone recovery, wherein the solution is an aqueous solution with pH 7.3-7.8, comprising:
1.80-2.06 g/L of 1-hydroxyethylidene diphosphonic acid, 1.44-2.22 g/L of anhydrous calcium chloride, 0.30-0.40 g/L of gadolinium (III) nitrate hexahydrate, and 0.038-0.076 g/L of dysprosium (III) chloride hexahydrate.
